(19) United States
(12) Patent Application Publication
(10) Pub. No.: US 2023/0197250 A1
(43) Pub. Date: Jun. 22, 2023

(54) MEDICAL INFORMATION PROCESSING DEVICE, MEDICAL INFORMATION PROCESSING METHOD, AND STORAGE MEDIUM

(71) Applicant: CANON MEDICAL SYSTEMS CORPORATION, Otawara-shi (JP)

(72) Inventors: Takuya SAKAGUCHI, Utsunomiya (JP); Ryo SHIROISHI, Nasushiobara (JP)

(73) Assignee: CANON MEDICAL SYSTEMS CORPORATION, Otawara-shi (JP)

(21) Appl. No.: 18/067,843

(22) Filed: Dec. 19, 2022

(30) Foreign Application Priority Data
Dec. 22, 2021 (JP) 2021-208115

(51) Int. Cl. G16H 30/40; G16H 30/20; G16H 10/40

(52) U.S. Cl. CPC G16H 30/40; G16H 10/40; G16H 30/20

(57) ABSTRACT

A medical information processing device includes a storage and processing circuitry. The storage stores past diagnostic images related to a subject. The processing circuitry is configured to acquire test result information on the subject and to identify a past diagnostic image associated with the test result information among the past diagnostic images stored in the storage.

### CROSS-REFERENCE TO RELATED APPLICATION

The present application claims priority based on Japanese Patent Application No. 2021-208115 filed Dec. 22, 2021, the content of which is incorporated herein by reference.

### FIELD

Embodiments disclosed in the present description and drawings relate to a medical information processing device, a medical information processing method, and a storage medium.

### BACKGROUND

In recent years, technological development has been promoted with the aim of improving the sensitivity of biological tests such as blood tests. In particular, a test called a liquid biopsy (LB) achieves highly sensitive detection. Such a blood test or the like can tell that a patient has an abnormality but cannot provide information about which organ or part of the patient has an abnormality. Although biomarkers that indicate which organ of a patient has an abnormality have emerged, which region of that organ is abnormal cannot be ascertained. Therefore, after a blood test, it is necessary to perform an imaging test to check for the presence or absence of a disease suspected due to the blood test, a degree of progression of the disease, and the location of the disease. Such a method requires that a new imaging test be requested if additional imaging tests are required. For this reason, such a method may result in exposure of a patient to radiation and additional test costs associated with imaging tests.

### BRIEF DESCRIPTION OF THE DRAWINGS

FIG. 1 is a diagram showing an example configuration of a test system according to a first embodiment.

FIG. 2 is a diagram showing the overview of a liquid biopsy method.

FIG. 3 is a diagram showing an example configuration of a test device of an embodiment.

FIG. 4 is a diagram showing an example configuration of a medical information processing device of an embodiment.

FIG. 5 is a diagram showing an example of a method of transmitting and receiving text result information between the test device and the medical information processing device according to an embodiment.

FIG. 6 is a flowchart of an example of a processing procedure of the test device according to an embodiment.

FIG. 7 is a flowchart of an example of a processing procedure of the medical information processing device according to the first embodiment.

FIG. 8 is a diagram showing an image search method according to an embodiment.

FIG. 9 is a diagram showing an operation example of the test system according to the embodiment.

FIG. 10 is a diagram showing an example configuration of a test system according to a second embodiment.

FIG. 11 is a diagram showing an example of an analysis model used in the second embodiment.

FIG. 12 is a diagram showing selection of an analysis model according to the second embodiment.

FIG. 13 is a flowchart of an example of a processing procedure of a medical information processing device according to the second embodiment.

FIG. 14 is a diagram showing an example of processing of a comparative example.

### DETAILED DESCRIPTION

Hereinafter, a medical information processing device, a medical information processing method, and a storage medium according to embodiments will be described with reference to the drawings.

A medical information processing device described in the embodiments includes a storage and processing circuitry. The storage stores past diagnostic images related to a subject. The processing circuitry is configured to acquire test result information on the subject and to identify a past diagnostic image associated with the test result information among the past diagnostic images stored in the storage. Accordingly, it is possible to facilitate imaging tests based on biological test results.

### First Embodiment

### Configuration of Test System

First, an example configuration of a test system will be described.

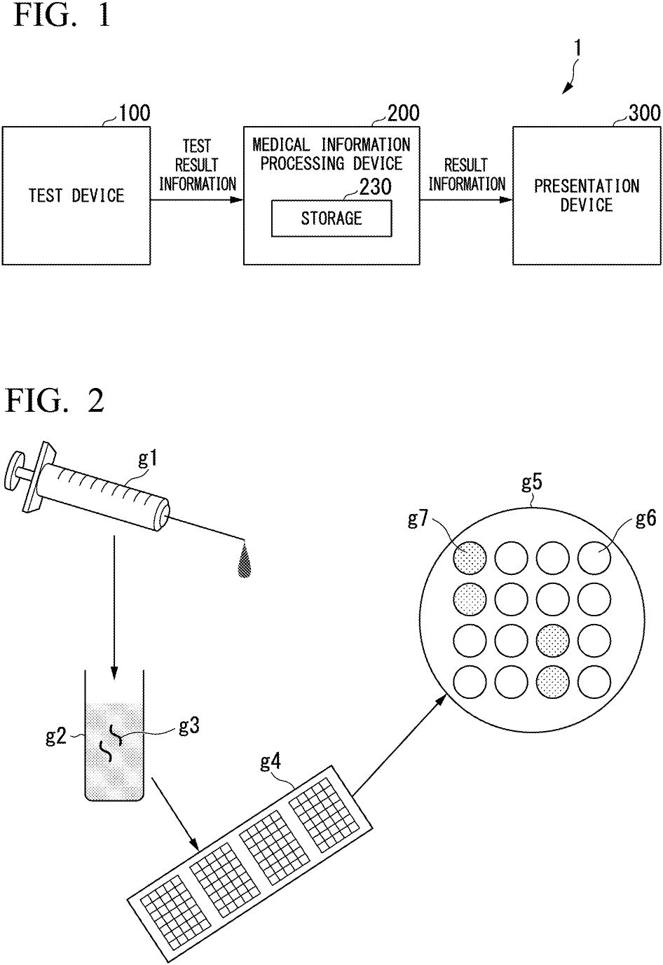

FIG. 1 is a diagram showing an example configuration of a test system according to the present embodiment. As shown in FIG. 1, the test system 1 includes, for example, a test device 100, a medical information processing device 200, and a presentation device 300. The test device 100 and the medical information processing device 200 are connected via a wireless or wired network.

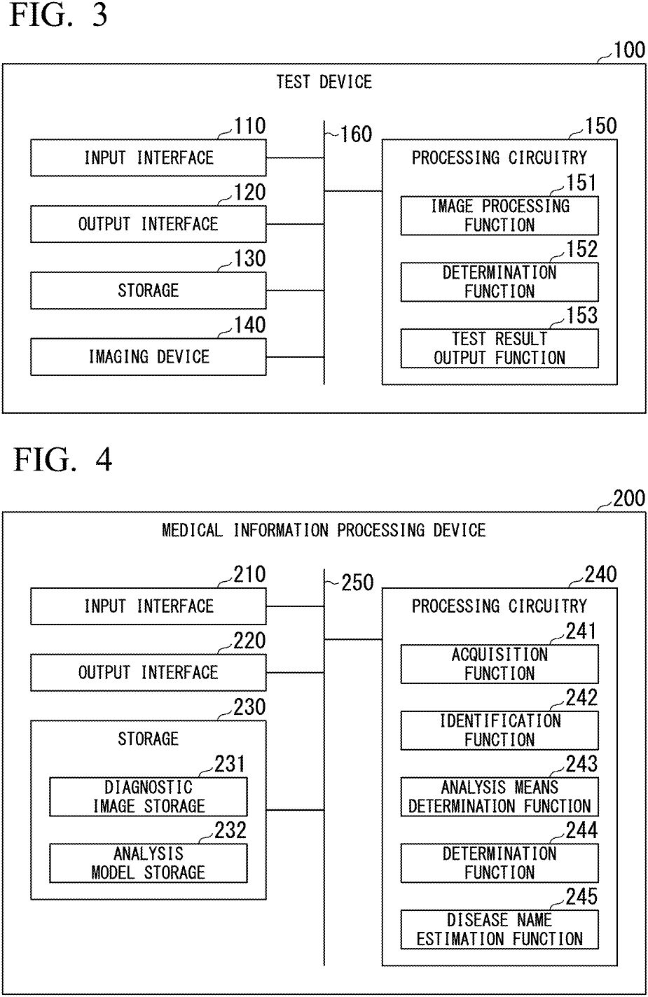

The test device 100 performs a test on a sample collected from a subject who is a person to be examined and outputs test result information based on test results to the medical information processing device 200. The test result information includes, for example, test result information about the subject. Although an example in which the test device 100 is a blood test device will be described in the following embodiments, the test device is not limited thereto. An example configuration and an example of processing of the test device 100 will be described later with reference to FIG. 3.

The medical information processing device 200 includes, for example, a storage 230. The medical information processing device 200 identifies a past diagnostic image associated with the test result information among information stored in the storage 230 on the basis of the test result information from the test device 100. The medical information processing device 200 outputs result information including the identified diagnostic image to the presentation device 300. An example configuration and an example of processing of the medical information processing device 200 will be described later with reference to FIG. 4.

The presentation device 300 is, for example, an image display device, a printing device, a tablet terminal, a smartphone, a mobile terminal, a dedicated terminal, or the like. The presentation device 300 presents the result information output by the medical information processing device 200.

### Example of Blood Test Method

Next, the overview of a liquid biopsy method will be described as an example of a blood test method used in the present embodiment. FIG. 2 is a diagram showing the overview of the liquid biopsy method. As shown in FIG. 2, first, blood is collected from a subject using, for example, a syringe g1. The blood collected from the subject may contain, for example, various tumor-derived components. Tumor-derived components g3 are, for example, circulating tumor RNA (ctRNA), circulating tumor DNA (ctDNA), tumor proteins, extracellular vesicles (exosomes), circulating tumor cells (CTC), tumor educated platelets (TEPs), etc.

In the liquid biopsy method, for example, genes extracted from the sample are labeled with a green fluorescent dye and a reference standard sample is labeled with a red fluorescent dye (g2 and g3). Next, in the liquid biopsy method, the labeled sample is subjected to a hybridization reaction with a DNA chip g4. After the reaction, the test device 100 reads the washed chip with a scanner (g5) and detects a signal. The signal is, for example, the ratio of red g7 (indicated by hatched circles in FIG. 2) to green g6 (indicated by non-hatched circles in FIG. 2). Accordingly, in the liquid biopsy method, it is possible to detect suspected diseases such as onset of cancer and tumors, for example. However, although it is possible to ascertain that the subject has an abnormality through the liquid biopsy method, it is not possible to ascertain which organ or site of the subject has the abnormality.

The blood test method is not limited to the liquid biopsy method. For example, a test method using a sample may be used.

### Configuration of Test Device

Next, an example configuration of the test device will be explained.

FIG. 3 is a diagram showing an example configuration of the test device of the present embodiment. As shown in FIG. 3, the test device 100 includes, for example, an input interface 110, an output interface 120, a storage 130, an imaging device 140, and processing circuitry 150. The input interface 110, the output interface 120, the storage 130, the imaging device 140, and the processing circuitry 150 are connected via a bus 160. The configuration shown in FIG. 3 is an example, and the configuration of the test device 100 is not limited thereto.

The input interface 110 receives various input operations from users and outputs operation information indicating results of the received input operations to the processing circuitry 150. The users may be, for example, doctors, nurses, pharmacists, clinical technologists, radiological technologists, physical therapists, and the like. In the present description, the input interface 110 is not limited to those including physical operation components such as a mouse and a keyboard. For example, examples of the input interface 110 also include electrical signal processing circuitry that receives an electrical signal corresponding to an input operation from an external input apparatus provided separately from the test device 100 and outputs the electrical signal to the control circuit.

The output interface 120 outputs information output by the processing circuitry 150 to the medical information processing device 200. The output interface 120 includes a communication circuit. In the present description, the output interface 120 is, for example, a transmission/reception terminal, a transmission/reception circuit, or the like. For example, examples of the output interface 120 also include electrical signal processing circuitry that outputs information or electrical signals to an external apparatus provided separately from the test device 100.

The storage 130 includes, for example, at least one of a read only memory (ROM), a random access memory (RAM), a hard disk drive (HDD), a solid state drive (SSD), and the like. The storage 130 stores, for example, programs used by the processing circuitry 150 for processing and control, threshold values, information necessary to determine the relationship between the ratio of red to green in a DNA chip and the name of a disease, and the like, mathematical formulas, address information of communication partners, identification information of communication partners, and the like.

The imaging device 140 images, for example, a DNA chip under the control of the processing circuitry 150. In the present description, the imaging device 140 is, for example, an imaging device including a complementary MOS (CMOS) imaging element, an imaging device including a charge coupled device (CCD) imaging element, or the like.

The processing circuitry 150 includes, for example, an image processing function 151, a determination function 152, and a test result output function 153. The processing circuitry 150 realizes these functions, for example, by a hardware processor executing a program stored in the storage 130 (storage circuit).

The hardware processor refers to circuitry such as a central processing unit (CPU), a graphics processing unit (GPU), an application specific integrated circuit (ASIC), a programmable logic device (for example, a simple programmable logic device (SPLD) or a complex programmable logic device (CPLD)), a field-programmable gate array (FPGA), or the like. Instead of storing the program in the storage 130, the program may be directly incorporated into a circuit of the hardware processor. In this case, the hardware processor reads and executes the program incorporated in the circuit to realize the functions. The hardware processor is not limited to being configured as a single circuit, and a plurality of independent circuits may be combined to be configured as a single hardware processor to realize each function. Further, a plurality of components may be integrated into one hardware processor to realize each function.

The image processing function 151 performs image processing on a determination image captured by the imaging device 140. For example, the ratio of red to green, the position of red, the position of green, and the like on a DNA chip are extracted through image processing.

The determination function 152 determines whether or not a blood test result is, for example, positive using an image processing result and information stored in the storage 130. Although an example of determining whether the result is positive or negative is described in the present embodiment, the determination is not limited thereto. For example, whether or not vital data is higher than a reference value, whether or not a liver γ-GTP value is higher than a reference value, or the like may be determined. When the determination result is positive, the determination function 152 estimates, for example, an organ having a corresponding abnormality, a candidate for the disease, and the like using the image processing result and the information stored in the storage 130. The determination function 152 outputs determination result information indicating the determination result to the test result output function 153. The determination result information includes, for example, information indicating whether the blood test result is positive or negative, information indicating whether or not vital data is higher than a reference value, information indicating whether the liver γ-GTP value is higher than a reference value, and the like.

The test result output function 153 outputs test result information to the medical information processing device 200 when the determination result satisfies predetermined conditions, such as when the blood test result is positive. The test result information includes, for example, determination result information, subject identification information, information indicating the name of a suspected disease, information indicating a site having a suspected disease, the name of an organ having a suspected disease, instructions for searching (or retrieving) past diagnostic images on the basis of determination result information, and the like.

### Configuration of Medical Information Processing Device

Next, an example configuration of the medical information processing device will be described.

FIG. 4 is a diagram showing an example configuration of the medical information processing device of the present embodiment. As shown in FIG. 4, the medical information processing device 200 includes, for example, an input interface 210, an output interface 220, the storage 230, and processing circuitry 240. The input interface 210, the output interface 220, the storage 230, and the processing circuitry 240 are connected via a bus 250. The configuration shown in FIG. 4 is an example, and the configuration of the medical information processing device 200 is not limited thereto. The medical information processing device 200 may be, for example, a workstation, a server, an image data server, a console device of a medical image diagnostic device, or the like. Further, the medical information processing device 200 may be provided in each hospital, for example, in the case of a hospital group or affiliated hospitals.

The input interface 210 receives various input operations from users and outputs operation information indicating results of the received input operations to the processing circuitry 240. The users may be, for example, doctors, nurses, pharmacists, clinical technologists, radiological technologists, physical therapists, and the like. In the present description, the input interface 210 is not limited to those including physical operation components such as a mouse and a keyboard. For example, examples of the input interface also include electrical signal processing circuitry that receives an electrical signal corresponding to an input operation from an external input apparatus provided separately from the device and outputs the electrical signal to the control circuit.

The output interface 220 causes the presentation device 300 to present result information, for example. In the present description, the output interface 220 is, for example, an image output circuit, an image output terminal, or the like. For example, examples of the output interface 220 also include electrical signal processing circuitry that outputs an image signal to the presentation device 300 provided separately from the medical information processing device 200.

The storage 230 includes, for example, at least one of a ROM, a RAM, an HDD, an SSD, and the like. The storage 230 stores, for example, programs, thresholds, mathematical formulas, address information of communication partners, identification information of communication partners, and the like used by the processing circuitry 240 for processing and control. The storage 230 also includes a diagnostic image storage 231 and an analysis model storage 232.

The diagnostic image storage 231 stores past diagnostic images regarding a plurality of subjects. Each past diagnostic image is associated with, for example, additional information such as a disease name, the type of diagnostic image, and site information. In addition, types of past diagnostic images include, for example, an image acquired by an X-ray computed tomography (CT) device, an image acquired by a magnetic resonance imaging (MRI) device, an image acquired by an ultrasonic diagnostic device, an image acquired by plain X-ray, and the like. Information indicating the type of a diagnostic image is associated with each diagnostic image. The diagnostic images stored in the diagnostic image storage 231 include, for example, past diagnostic images of a subject, past diagnostic images of other subjects, diagnostic images for diseases, diagnostic images when healthy, and the like.

The analysis model storage 232 stores a plurality of analysis models. Analysis models will be described later.

The processing circuitry 240 includes, for example, an acquisition function 241, an identification function 242, an analysis means determination function 243, a determination function 244, and a disease name estimation function 245. The processing circuitry 240 realizes these functions by, for example, a hardware processor executing a program stored in the storage 230 (storage circuit). The processing circuitry 240 outputs result information including an identified past diagnostic image to the presentation device 300 via the output interface 220.

Instead of storing the program in the storage 230, the program may be directly incorporated into the circuit of the hardware processor. In this case, the hardware processor realizes the functions by reading and executing the program incorporated in the circuit. The hardware processor is not limited to being configured as a single circuit and may be configured as one hardware processor by combining a plurality of independent circuits to realize each function. Further, a plurality of components may be integrated into one hardware processor to realize each function.

The acquisition function 241 acquires test result information output by the test device 100.

The identification function 242 identifies at least one of the past diagnostic images stored in the diagnostic image storage 231 on the basis of the test result information acquired by the acquisition function 241 in a case where identification is possible. Alternatively, the identification function 242 identifies a past diagnostic image using an analysis model on the basis of site information determined by the determination function 244. The identification function 242 outputs result information including the identified past diagnostic image to the presentation device 300 via the output interface 220.

The analysis means determination function 243 determines an analysis model for the past diagnostic image identified by the identification function 242 from among a plurality of analysis models stored in the analysis model storage 232 on the basis of the test result information acquired by the acquisition function 241.

The determination function 244 determines site information indicating a test site of the subject on the basis of the test result information.

The disease name estimation function 245 estimates a disease name of the subject on the basis of site information associated with past diagnostic images.

### Example of Method of Transmitting and Receiving Test Result Information

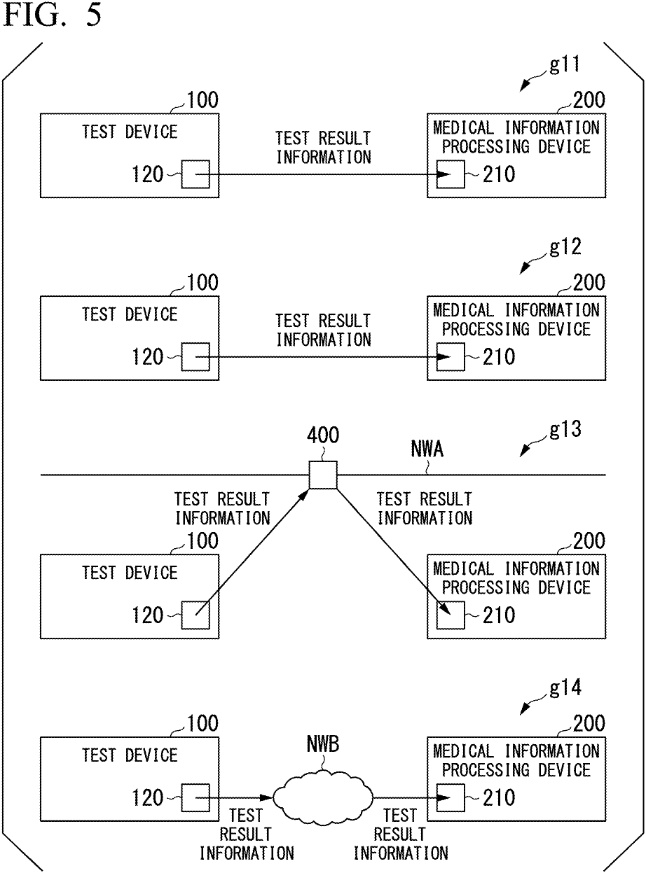

Next, an example of a method of transmitting and receiving test result information between the test device 100 and the medical information processing device 200 will be described. FIG. 5 is a diagram showing an example of a method of transmitting and receiving test result information between the test device and the medical information processing device according to the present embodiment.

In a first example (g11), the test device 100 automatically transmits test result information to the medical information processing device 200 via the output interface 120 when a blood test result is positive. The medical information processing device 200 receives the test result information via the input interface 210.

In a second example (g12), a user of the test device 100, for example, operates an operation button or a touch panel included in the input interface 110 of the test device 100 when a blood test result is positive. The test device 100 transmits test result information to the medical information processing device 200 via the output interface 120 on the basis of the operation result. The medical information processing device 200 receives the test result information via the input interface 210.

In a third example (g13), the test device 100 transmits test result information to, for example, a repeater 400 on a wired network NWA via the output interface 120 when a blood test result is positive. Meanwhile, transmission may be automatic or may be based on a user operation. The repeater 400 transmits the test result information to the medical information processing device 200. The medical information processing device 200 receives the test result information via the input interface 210.

In a fourth example (g14), the test device 100 transmits test result information to the medical information processing device 200 through a network NWB via the output interface 120 when a blood test result is positive. Meanwhile, transmission may be automatic or may be based on a user operation. The medical information processing device 200 receives the test result information via the input interface 210.

The method of transmitting and receiving test result information shown in FIG. 5 is an example and is not limited thereto.

### Example of Processing Procedure of Test Device

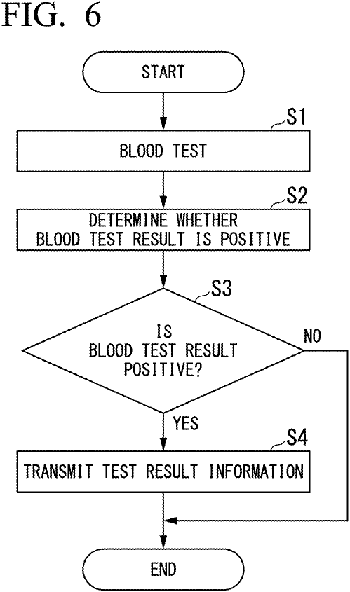

Next, an example of a processing procedure of the test device 100 will be described. FIG. 6 is a flowchart of an example of the processing procedure of the test device according to the present embodiment.

The test device 100 tests blood collected from a subject (step S1). As a timing at which blood is acquired, for example, the time of medical examination, or the like is conceivable. The image processing function 151 performs image processing on, for example, a determination image obtained by imaging a DNA chip.

The determination function 152 determines whether the blood test result is positive on the basis of the result of image processing performed on the determination image (step S2). If the blood test result is positive, the determination function 152 transmits test result information to the medical information processing device 200 (steps S3 and S4). If the blood test result is not positive, determination function 152 ends processing (step S3).

The processing and procedure shown in FIG. 6 are an example and are not limited thereto.

### Example of Processing Procedure of Medical Information Processing Device

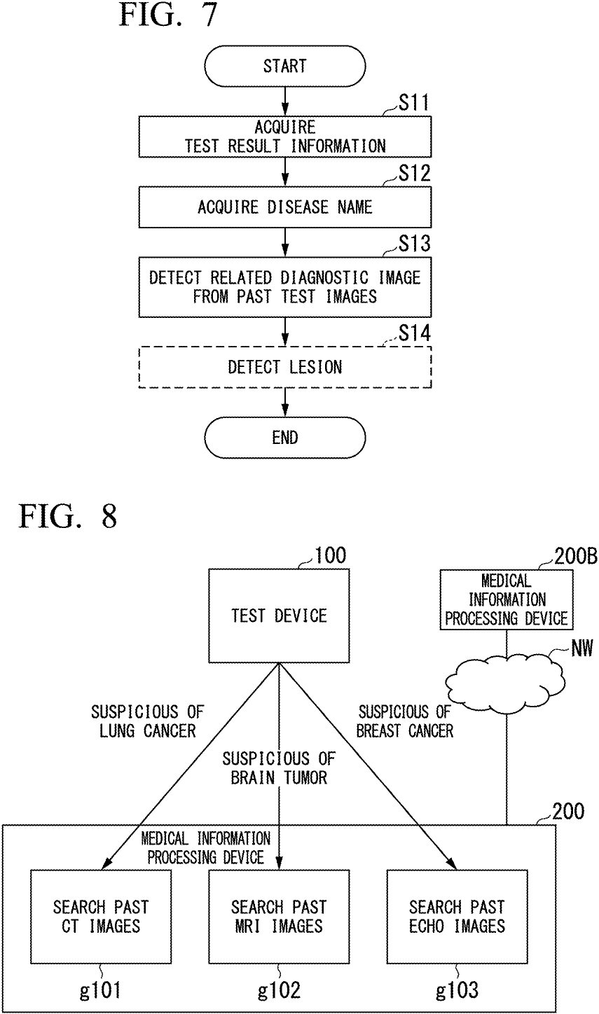

Next, an example of a processing procedure of the medical information processing device 200 will be described. FIG. 7 is a flowchart of an example of the processing procedure of the medical information processing device according to the present embodiment.

The acquisition function 241 acquires test result information transmitted by the test device 100 via the input interface 210 (step S11). The acquisition function 241 acquires a disease name included in the test result information (step S12).

The identification function 242 identifies at least one of past diagnostic images stored in the diagnostic image storage 231 on the basis of the test result information acquired by the acquisition function 241 in a case where identification is possible (step S13). The case where identification is possible is, for example, a case where the corresponding diagnostic image has been acquired in the past and the corresponding diagnostic image is stored in the diagnostic image storage 231.

The medical information processing device 200 may also perform the following processing. The disease name estimation function 245 detects a lesion on the basis of the identified past diagnostic image (step S14).

The processing and procedure shown in FIG. 7 are an example and are not limited thereto.

### Image Search Method

An image search method will be described. FIG. 8 is a diagram showing an image search method according to the present embodiment. In the present embodiment, test result information transmitted by the test device 100 includes the name of a suspected disease, the name of an organ having a suspected disease, and the like, as described above. Therefore, as shown in FIG. 8, when the medical information processing device 200 acquires “suspicious of lung cancer” from the test device 100, the medical information processing device 200 searches past diagnostic images acquired using a CT device and determines past lung field diagnostic images using the CT device. When the medical information processing device 200 acquires “suspicious of brain tumor” from the test device 100, the medical information processing device 200 searches past diagnostic images acquired using an MRI device and determines past brain diagnostic images acquired using the MRI device. When the medical information processing device 200 acquires “suspicious of breast cancer” from the test device 100, the medical information processing device 200 searches past diagnostic images acquired using an ultrasonic diagnostic device and determines past chest diagnostic images acquired using the ultrasonic diagnostic device. In this manner, search targets for past diagnostic images are switched according to test results in the present embodiment. In the present embodiment, a search target is determined on the basis of test results of a selected test item. For example, if a blood test result is “presence of pancreatic cancer,” test result information including a search instruction is transmitted such that past diagnostic images of the pancreatic cancer are searched. The example shown in FIG. 8 is an example and is not limited thereto.

As described above, in the present embodiment, rules may be defined in advance in a table for past diagnostic images set to targets as a result of a blood test. Further, the medical information processing device 200 may change the target and method of a past diagnostic image to be targeted according to a degree of test result as a result of a blood test. For this reason, the medical information processing device 200 may have a function of classifying the seriousness of a disease into high, medium, or low degree or classify it by a threshold value from blood test results.

The medical information processing device 200 may change a test range such that only diagnostic images in the corresponding hospital are searched as a past diagnostic image search target range, for example, if a mild disease is suspected, and past diagnostic images stored in a medical information processing device 200B of a group hospital connected via a network NW are searched, as shown in FIG. 8, in case of a severe disease, for example.

Alternatively, the medical information processing device 200 may change a search target period depending on the severity of a disease in such a manner that it searches the past one year as the past diagnostic image search target range, for example, if a mild disease is suspected, and searches the past ten years in case of a severe disease, for example.

The medical information processing device 200 may search past diagnostic images with a high priority when the urgency of a disease suspected as a result of a blood test is high and perform the search at night or on a holiday when the urgency is low. The urgency is stored in the storage 130 of the test device 100 or the storage 230 of the medical information processing device 200, for example.

Furthermore, when a blood test result is positive, the medical information processing device 200 searches not only related organs but also related diseases, for example, from past diagnostic images. For example, the medical information processing device 200 may also search past diagnostic images of potentially metastatic bones, brains, livers, and adrenal glands when a liquid biopsy test result for lung cancer is positive.

### Operation Example

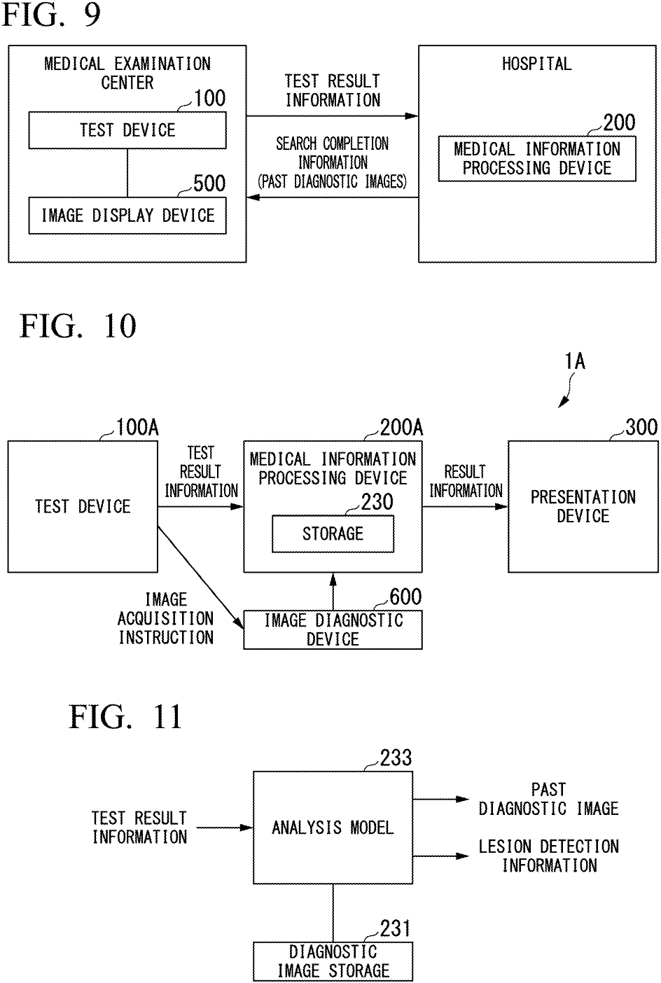

Here, an operation example of the test system 1 will be explained. FIG. 9 is a diagram showing an operation example of the test system according to the present embodiment. As shown in FIG. 9, for example, the test device 100 is installed in a medical examination center for medical examinations and the medical information processing device 200 is installed in a hospital.

In a medical examination, for example, a blood test and a genetic test are performed on a subject. Accordingly, it is desirable that past diagnostic image search results be available at the stage of explaining blood test results to the subject.

Therefore, the test device 100 transmits test result information to the medical information processing device 200. The medical information processing device 200 transmits search completion information indicating that search of past diagnostic images has ended to the test device 100. It is desirable that the medical information processing device 200 search past diagnostic images before explaining the test results to the subject at the medical examination center. Then, the test device 100 causes an image display device 500 connected to the test device 100, for example, to display an image (for example, an icon, a text image, etc.) indicating completion of search. This information may be linked to an electronic medical record, for example.

The medical information processing device 200 may be installed in a hospital to which test result information is transmitted from the medical examination center or may be installed in a group hospital of the hospital. Information transmitted from the medical examination center may be an electronic referral letter or the like including test result information. Further, the diagnostic image storage 231 may not be installed in the hospital equipped with the medical information processing device 200. In this case, information stored in the diagnostic image storage 231 may be stored in another location or in a cloud. Furthermore, the medical information processing device 200 or the test device 100 may transmit a search request to a medical institution to which the subject has a history of visits to acquire past diagnostic images. In this case, past diagnostic images are stored in the medical institution to which the subject has a history of visits. The test device 100 may acquire information on the medical institution to which the subject has a history of visits from, for example, the subject’s health insurance card or an application installed in a smartphone or the like. In addition, the medical information processing device 200 may transmit past diagnostic images of a site of interest related to a blood test result of the liquid biopsy method to the test device 100. A medical center or a hospital may transmit diagnostic results to the subject.

For example, the test device 100 or the medical information processing device 200 may display past diagnostic images after automatically performing preprocessing suitable for the site of interest related to the liquid biopsy method. For example, the medical information processing device 200 may execute preprocessing of generating a pancreatic duct cross-sectional image from a liver test image through image processing to generate the image when a blood test result of the liquid biopsy method with respect to pancreatic duct cancer is positive. The medical information processing device 200 may not display past diagnostic images of test for different purposes as they are.

As described above, when a subject is suspected of having a disease through a blood test, test result information including a search request for past diagnostic images related to the disease among diagnostic images captured in the past is transmitted to the medical information processing device 200 in the present embodiment. In this manner, a search request (image analysis request or the like) for past tests is transmitted to the medical information processing device 200 such that the past tests are checked in the present embodiment.

Therefore, according to the present embodiment, it is possible to facilitate imaging test based on biological test results.

Although an example of a blood test has been described in the above test example, it is not limited thereto. The test may be, for example, a sputum test, runny nose/mucous membrane, stool test, urine test, or the like.

### Second Embodiment

Hereinafter, a second embodiment will be described. The second embodiment differs from the first embodiment in that an analysis model is used to search past diagnostic images. Therefore, the following description will focus on differences from the first embodiment, and the description of points that are common to the first embodiment will be omitted.

### Configuration of Test System

First, an example configuration of a test system of the present embodiment will be described.

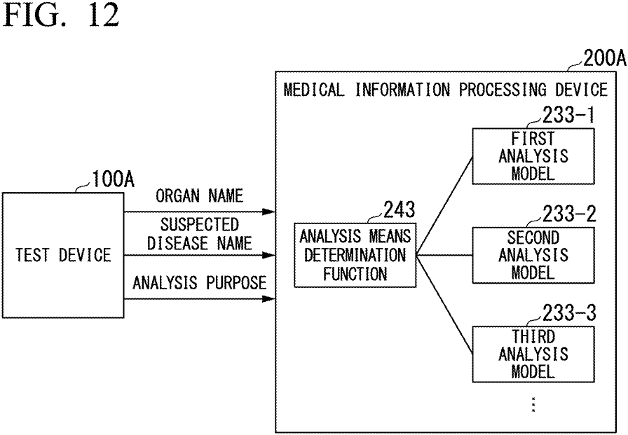

FIG. 10 is a diagram showing an example configuration of the test system of the present embodiment. As shown in FIG. 10, the test system 1A includes, for example, a test device 100A, a medical information processing device 200A, a presentation device 300, and an image diagnostic device 600. The test device 100A and the medical information processing device 200A are connected via a wireless or wired network. The medical information processing device 200A and the image diagnostic device 600 are connected via a wireless or wired network.

The configuration of the test device 100A is the same as that of the test device 100 of the first embodiment. The configuration of the medical information processing device 200A is the same as that of the medical information processing device 200 of the first embodiment.

The test device 100A transmits an image acquisition instruction to the image diagnostic device 600 when test result information including a test request has been transmitted to the medical information processing device 200A.

The medical information processing device 200A searches and selects past diagnostic images using an analysis model according to the test result information transmitted by the test device 100A. The medical information processing device 200A acquires diagnostic images output by the image diagnostic device 600 and processes the acquired diagnostic images.

The image diagnostic device 600 is, for example, an X-ray CT device, an MRI device, an ultrasonic diagnostic device, a plain roentgenography device, a nuclear medicine diagnostic device, or the like. The image diagnostic device 600 performs, for example, a test using an X-ray CT device, a test using an MRI device, a test using an ultrasonic diagnostic device, a test using a plain roentgenography device, or the like on the subject according to the image acquisition instruction transmitted by the test device 100A.

### Analysis Model

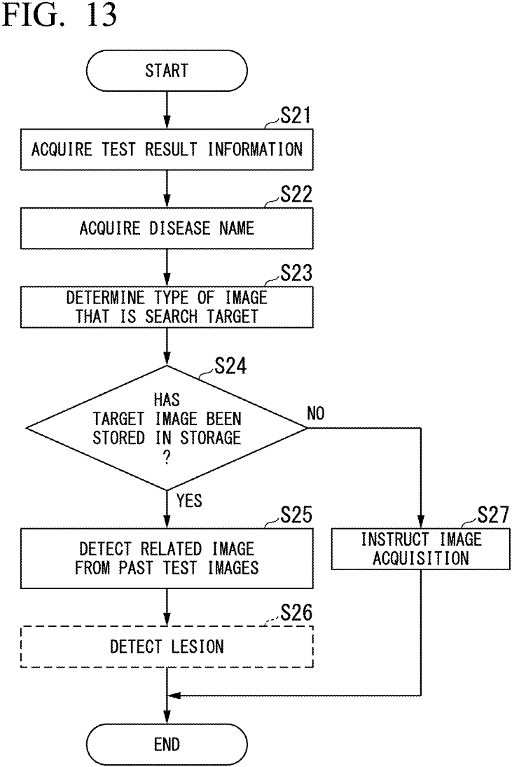

Here, an example of the analysis model used in the present embodiment will be described. FIG. 11 is a diagram showing an example of the analysis model used in the present embodiment. The analysis model 233 receives test result information and outputs past diagnostic images associated with the test result information. Further, the analysis model 233 receives test result information and outputs lesion detection information. The analysis model is generated by inputting teacher data in advance, which is a set of test result information and past diagnostic images, and training the teacher data. Further, the analysis model is generated by inputting teacher data in advance, which is a set of test result information and lesion detection information, and training the teacher data.

There is a plurality of analysis models 233. The respective analysis models 233 are models separately trained for respective image types (an image acquired using a CT device, an image acquired using an MRI device, an image acquired using an ultrasonic diagnostic device, and an image acquired using plain X-rays), for example. For example, the analysis model 233 for images acquired using a CT device is trained using images acquired using the CT device and searches for images acquired using the CT device at the time of searching. Further, the analysis models 233 may be models separately trained for respective diseases (lung cancer, breast cancer, liver cancer, colon cancer, brain tumor, and the like). Further, the analysis models 233 may be models trained for respective organs or diseases, and for example, the analysis model 233 for lung cancer is trained using images of lung cancer and the name of the disease and searches for diagnostic images showing lung fields. In addition, the analysis models 233 may perform training by combining the above-described training units.

FIG. 12 is a diagram showing selection of an analysis model according to the present embodiment. In the example of FIG. 12, test result information transmitted by the test device 100A includes, for example, the name of an organ, the name of a suspected disease, the purpose of analysis, and the like. The purpose of analysis is, for example, “ascertaining the presence or absence of a disease,” “ascertaining the location of a disease,” “ascertaining the severity of a disease,” or the like.

When the analysis purpose acquired from the test device 100A is “ascertaining the presence or absence of a disease,” the analysis means determination function 243 selects, for example, a first analysis model 233-1 that is highly sensitive to the presence or absence of a disease.

When the analysis purpose acquired from the test device 100A is “ascertaining the location of a disease,” the analysis means determination function 243 selects, for example, a second analysis model 233-2 that is highly sensitive to the location of a disease.

When the analysis purpose acquired from the test device 100A is “ascertaining the severity of a disease,” the analysis means determination function 243 selects, for example, a third analysis model 233-3 that is highly sensitive to characteristic (severity) analysis.

For example, if the analysis purpose is “ascertaining the location of a disease,” no emphasis is placed on the accuracy of the presence or absence of a disease, and thus it is desirable to return a result “if there is a disease, here.” In this case, the medical information processing device 200A returns, to the test device 100A, information indicating, for example, a place where a part of the corresponding organ has a strange shape. This analysis model may not consider whether or not there is a disease in the returned information.

Further, the medical information processing device 200A may add a comment to a search result and return it to the test device 100A. The added comment is, for example, “past diagnostic images were too old,” “target organs were missing from the images,” “images were present but not contrast-imaged,” “images were present but the energy or sequence is different,” or the like. This function is necessary because the current analysis target is not searched from diagnostic images newly captured for the purpose of this disease test but is searched from diagnostic images captured in the past.

Meanwhile, the analysis purpose and the analysis models 233 described using FIG. 12 are an example, and are not limited thereto. For example, the analysis models 233 may also include a general-purpose analytic model. In this case, the processing circuitry 240 may also use an analysis model that is suitable for the analysis purpose or a diseased organ (highly sensitive to the organ) along with the general-purpose analysis model.

### Example of Processing Procedure of Medical Information Processing Device

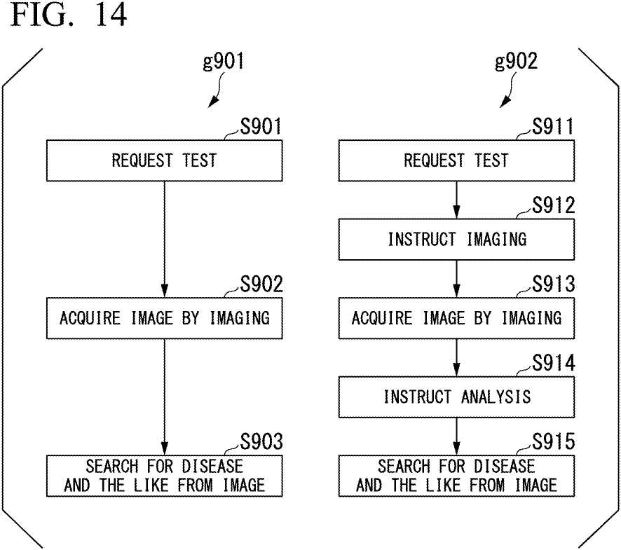

Next, an example of a processing procedure of the medical information processing device 200A will be described. FIG. 13 is a flowchart of an example of the processing procedure of the medical information processing device according to the present embodiment.

The acquisition function 241 acquires test result information transmitted by the test device 100A via the input interface 210 (step S21). The acquisition function 241 acquires a disease name included in the test result information (step S22).

The determination function 244 determines target site information and the type of a past diagnostic image that is a search target on the basis of the test result information (step S23).

The determination function 244 determines whether or not the target past diagnostic image is stored in the diagnostic image storage 231 on the basis of the determined image type of the search target (step S24).

If the past diagnostic image of the target is stored in the diagnostic image storage 231, the analysis means determination function 243 determines at least one analysis model from among the plurality of analysis models stored in the analysis model storage 232 on the basis of the test result information, the type of the past diagnostic image that is the search target, and the site information. The identification function 242 identifies a past diagnostic image of a target location using the analysis model on the basis of the site information determined by the determination function 244 (step S25).

The disease name estimation function 245 may detect a lesion on the basis of the past diagnostic image of the target location (step S26).

When the diagnostic image storage 231 does not store the target past diagnostic image, the processing circuitry 240 outputs an image acquisition instruction to the test device 100A. The processing circuitry 240 may output an imaging instruction to the image diagnostic device 600.

The processing and procedures shown in FIG. 13 are an example, and are not limited thereto.

Therefore, according to the present embodiment, a past diagnostic image associated with test result information can be acquired using an analysis model. Furthermore, even if there is no past diagnostic image, a test for new image acquisition can be performed and necessary diagnostic images can be acquired.

### Comparative Examples

Here, an example of processing comparative examples will be described. FIG. 14 is a diagram illustrating an example of processing comparative examples.

### First Comparative Example (g901)

In test processing, when a request is issued (step S901), an image is captured first (step S902), image analysis is performed on the captured diagnostic image, and disease search is performed (step S903).

### Second Comparative Example (g902)

In test processing, when a request is issued (step S911), an imaging instruction is issued (step S902), a diagnostic image is captured (step S913), an image analysis instruction is issued for the captured diagnostic image (step S914), and disease search is performed from the diagnostic image (step S914).

When only image analysis is performed without image capture, it may be conceived that a doctor consciously has a different mindset when searching for a disease from new diagnostic images and searching for a disease from past diagnostic images.

If an analysis model is used to search for a disease from new diagnostic images, which organ or site has an abnormality or a disease is not ascertained in many cases. Or the accuracy is not so high. For example, for a subject who has a headache, a diagnostic image of the head is captured and the head is analyzed. In such a case, the name of the disease is often unknown. Alternatively, when a follow-up examination for cancer is performed after half a year, it is possible to examine whether the cancer has grown or shrunk and whether or not there is metastasis by comparing diagnostic images captured in the past with currently captured diagnostic images.

### Comparison Between Embodiment and Comparative Example

In search for past diagnostic images after a blood test, as in the embodiment, which organ or site has cancer is ascertained through the blood test. Therefore, diagnostic image search specialized for a target organ is performed in the embodiment, and thus search can be performed with high accuracy.

Here, the analysis model of the present embodiment will be further described. In the present embodiment, the analysis model 233, which is a search engine for past diagnostic images and is different from a search engine for general images, is used. In addition, in the present embodiment, search is performed even if a past diagnostic image that is a search target is not a diagnostic image captured exclusively for a target disease. Accordingly, search parameters are different from those used in search for general images in the present embodiment. Furthermore, in the present embodiment, the analysis model uses a search engine specialized for searching past diagnostic images, unlike those for general images.

In the present embodiment, when the pancreas is found to be bad through a blood test, the pancreas is found from diagnostic images captured in the past even if the diagnostic images are not diagnostic images captured for the liver, and pancreatic cancer is detected. At the time of searching, an instruction is transmitted from the test device 100 (or 100A) such that an analysis model for pancreatic cancer detection instead of an analysis model for liver cancer detection is selected. In the present embodiment, even if identification information (a tag, purpose of examination, and medical record order) of liver examination is included in test result information, pancreas diagnostic images are searched. That is, in the present embodiment, diagnostic images of organs other than an organ to be tested are also searched. The processing circuitry 240 of the medical information processing device 200 (or 200A) searches and selects a past diagnostic image even if it is a diagnostic image in which, for example, only half or part of the pancreas is shown.

For example, in a pancreatic cancer examination, it is common to find pancreatic cancer even using contrast-imaged diagnostic images. However, past diagnostic images also include diagnostic images in which the pancreas is shown without contrast imaging even if pancreas contrast imaging has not been performed. Therefore, in the present embodiment, pancreatic cancer is also searched for using non-contrast-imaged images. In the present embodiment, for training an analysis model, a currently printed diagnostic image may be used, or an engine trained using non-contrast-imaged images may be used for searching for past diagnostic images. In this case, it is desirable to switch analysis models 233 to be used for search, as described above.

When the presence or absence of cancer is searched using a current diagnostic image, not only sensitivity but also specificity are important. This is because misdiagnosing a person who does not have cancer as having cancer will increase medical expenses and burden on the patient. For this reason, it is desirable to use the analysis model 233 that is optimized to balance both sensitivity and specificity. In a blood test as in the present embodiment, cancer has already been found through the blood test. Therefore, in the present embodiment, it is not necessary to consider “erroneously determining cancer,” and it is required to positively identify a cancerous region. As described above, in the present embodiment, it is desirable to use an engine optimized to search for past diagnostic images as the analysis model 233 used to search for diagnostic images.

Alternatively, if the characteristics of the blood test are too sensitive, the sensitivity of image search parameters may be curbed and adjusted to increase specificity.

Further, in the present embodiment, an analysis purpose can be input to the medical information processing device 200 (or 200A), as described above. In the present embodiment, it is possible to switch an image search engine or switch parameters of an analysis model, which is the image search engine, depending on the purpose expected from past diagnostic image search. Further, as described above, an analysis purpose may be designated as “searching for the presence or absence of a disease,” “searching for the location of a disease,” “searching for the severity of a disease,” or the like.

Furthermore, such purposes may be determined automatically on the basis of test results of the liquid biopsy method. For example, if the result of a blood test is low in accuracy, a purpose is set to search for “presence/absence” in an image test as well. For example, if the probability of a blood test is high, it is not necessary to determine the presence or absence of a disease, and rather, the purpose may be set to search for past diagnostic images of a diseased organ or site. In this manner, it is possible to identify changes in organs, to search for abnormal sites, to search for changes in organs to search for an organ or a site that has (or is likely to have) an abnormality, or to find changes in characteristics of an organ, instead of searching for past diagnostic images of cancer, for example, in the present embodiment.

Further, in the present embodiment, results unique to past diagnostic image search may be returned to the test device 100, as described above. For example, there are cases that are impossible for diagnostic images captured for the purpose of pancreatic examination. In this case, the following information may be returned to the test device 100, for example. In addition, the following example is an example, and is not limited thereto.
I. “The pancreas was not shown.”
II. “There is a pancreas image, but it was 10 years old.”
III. “Only ⅓ of the pancreas was shown and ⅔ was missing.”
IV. “The accuracy is like to be low due to searching on non-contrast-imaged images.”
V. “Searching was performed with different imaging energies and sequences form those for the pancreas.”

In addition, the test time differs between a test using the liquid biopsy method and a test using past diagnostic images. Even when symptoms of a subject are severe at the time of testing using the liquid biopsy method, the symptoms are likely to be mild at the time of past testing. For this reason, in the present embodiment, the parameters of the analysis model 233 are adjusted according to a time interval between a test by the liquid biopsy method and a test using past diagnostic images. For example, a detection threshold value of the analysis model 233 may be set to 0.8 for diagnostic images half a year ago and 0.5 for diagnostic images one year ago. The value of the detection threshold value is an example, and is not limited thereto.

As described above, in the present embodiment, selection of past diagnostic images, and the like are performed using a trained analysis model. Further, in the present embodiment, a plurality of analysis models are prepared and selected according to analysis purpose. In addition, in the present embodiment, past diagnostic image search results are returned to the test device 100.

Therefore, according to the present embodiment, it is possible to acquire past diagnostic images according to an analysis purpose to perform diagnosis using the acquired past diagnostic images.

According to at least one embodiment described above, it is possible to facilitate imaging test based on biological test results by including the storage 230, the acquisition function 241, and the identification function 242.

Although several embodiments have been described, these embodiments are presented as examples and are not intended to limit the scope of the invention. These embodiments can be implemented in various other forms, and various omissions, substitutions, and modifications can be made without departing from the scope of the invention. These embodiments and modifications thereof are included in the scope and spirit of the invention, as well as the scope of the invention described in the claims and equivalents thereof.

What is claimed is:

1. A medical information processing device comprising:
a storage stores past diagnostic images related to a subject; and
processing circuitry configured to acquire test result information on the subject and to identify a past diagnostic image associated with the test result information among the past diagnostic images stored in the storage.

2. The medical information processing device according to claim 1, wherein the storage further stores a plurality of analysis models, and the processing circuitry determines an analysis model for the identified past diagnostic image from among the plurality of analysis models stored in the storage on the basis of the test result information.

3. The medical information processing device according to claim 1, wherein the processing circuitry determines site information indicating a test site of the subject on the basis of the test result information and identifies the associated past diagnostic image further on the basis of the site information.

4. The medical information processing device according to claim 2, wherein the processing circuitry determines site information indicating a test site of the subject on the basis of the test result information and determines the analysis model further on the basis of the site information.

5. The medical information processing device according to claim 1, wherein the test result information to be acquired is test result information related to a liquid biopsy method.

6. The medical information processing device according to claim 1, wherein the processing circuitry identifies the past diagnostic image associated with the test result information by analyzing the past diagnostic images stored in the storage.

7. The medical information processing device according to claim 1, wherein the processing circuitry identifies the past diagnostic image associated with the test result information by analyzing supplementary information of the past diagnostic images stored in the storage.

8. The medical information processing device according to claim 1, wherein the processing circuitry outputs the identified past diagnostic image.

9. A medical information processing method, performed by a medical information processing device, comprising:
acquiring test result information on a subject; and
identifying a past diagnostic image associated with the test result information from past diagnostic images related to the subject and stored in a storage.

10. A computer-readable non-transitory storage medium storing a program for causing a computer to:
acquire test result information on a subject; and
identify a past diagnostic image associated with the test result information from past diagnostic images related to the subject and stored in a storage.
